(19) United States
(12) Patent Application Publication
(10) Pub. No.: US 2024/0302278 A1
(43) Pub. Date: Sep. 12, 2024

(54) A CYNADIDE DETERMINATION SYSTEM AND A METHOD FOR OBTAINING GOLD NANOCLUSTERS USED IN THE SYSTEM

(71) Applicant: HACETTEPE UNIVERSITESI REKTORLUK, Cankaya/Ankara (TR)

(72) Inventors: Ugur AYDIN, Ankara (TR); Memed DUMAN, Ankara (TR)

(73) Assignee: HACETTEPE UNIVERSITESI REKTORLUK, Cankaya/Ankara (TR)

(21) Appl. No.: 18/552,402

(22) PCT Filed: Mar. 31, 2022
(86) PCT No.: PCT/TR2022/050280

§ 371 Date: Sep. 25, 2023

(30) Foreign Application Priority Data
Mar. 31, 2021 (TR) 2021/005815

(51) Int. Cl. G01N 21/64; G01N 1/40

(52) U.S. Cl. CPC G01N 21/6428; G01N 1/4077; G01N 21/6456; G01N 2001/4083; G01N 2021/6439

(57) ABSTRACT

The present invention relates to a system which enables to carry out cyanide determination in biological samples taken from the living beings who are exposed to cyanide gas and/or cyanide components especially released from the industry and/or house fires, and a method of obtaining gold nanoclusters used in the system.

### TECHNICAL FIELD

The present invention relates to a system which enables to carry out cyanide determination in biological samples taken from the living beings who are exposed to cyanide gas and/or cyanide components especially released from the industry and/or house fires, and a method for obtaining gold nanoclusters used in the system.

### BACKGROUND OF THE INVENTION

Cyanide can be found in many household foods and products, and it is particularly released as combustion products of many synthetic materials used in the industry as well. In particular, it is stated in the literature that it is not easy to make distinction between the hydrogen cyanide (HCN) gas poisoning and carbon-monoxide (CO) poisoning due to combustion of components containing cyanide in many industry and house fires, there are difficulties in diagnosis and implementation of the related treatment and this could result in great loss of life.

Both carbon-monoxide and cyanide poisoning may cause a combination of gastrointestinal disorders, including nausea and vomiting, and neurological signs such as dizziness and headache to some extent. These findings are generally seen in mild to moderate carbon-monoxide poisonings. In addition, it is reported that these findings are seen in the early stage of cyanide poisoning as well. In carbon-monoxide poisoning, Normobaric Oxygen Therapy (NBO) and/or Hyperbaric Oxygen Therapy (HBO) is administered to patients. Whereas in cyanide treatment, determining the cyanide analytically before an antidotal therapy ensures that evaluation of the severity of poisoning and antidote dose levels can be carried out more accurately. Antidotes can also be toxic, especially at high doses.

Today, devices such as conventional spectrochemical absorption or luminescence methods, electrochemical methods, GC-NPD (GC with Nitrogen Phosphorous Detector) and GC-MS (Gas Chromatography Mass Spectrometry) with capillary electrophoresis and gas or liquid chromatography techniques based on various detection techniques are used in order to detect the cyanide in biological samples such as blood. Devices used for the cyanide determination have extremely high costs and include complex and sensitive components that must be used by a specialist personnel. Thus, they are not suitable for use by first response personnel to obtain quick and precise results for diagnosis. Also, since they are expensive, it cannot be ensured that these devices are available in every hospital. In this case, very long period of times are required to send the blood samples taken for cyanide detection to the nearest hospitals having the necessary test devices, and to decide on the treatment method to be applied by detecting the cyanide and finding out the results. However, many toxic effects occur as a result of oral ingestion, inhalation and absorption through the skin of cyanide-containing substances. Since the half-life of cyanide is short, the speed of diagnosis is also of great importance for treatment. Today, cyanide determination studies carried out on the basis of blood and urine are insufficient in terms of both sample storage and transportation, cost and analysis periods.

Therefore, there is a need for a system which enables to evaluate a biological sample taken from a person who is exposed to cyanide in a very short time and to measure the concentration of exposed cyanide by means of an easily accessible, low-cost and simple-to-use method.

The Chinese patent document no. CN105842181A, an application in the state of the art, discloses a method for detecting cyanide ions by means of gold nanorods. More particularly, the method is based on the characteristics that gold nanorods react with the cyanide ion when they contact with a sample containing a cyanide ion and changes are detected in its optical properties. Accordingly, detection of cyanide in a liquid compound containing cyanide ions can be performed quickly and precisely. In the method, a gold nanorod solution is prepared by using HAuCl₄ at first and unwanted components included in the gold nanorod solution are separated by centrifugation. Then, a working curve is created according to the ultraviolet spectrum values of the reaction which is performed by combining a series of cyanide ion standard solutions and gold nanorod solution. Thereafter, a measurement is carried out for the samples and the concentration of the cyanide ion is calculated by comparing the spectrum values obtained for the reaction result upon adding the gold nanorod solutions into the sample with the working curve.

### SUMMARY OF THE INVENTION

An objective of the present invention is to realize a system which enables to carry out cyanide determination in biological samples taken from the living beings who are exposed to cyanide gas or cyanide components released from the industry or house fires, and a method for obtaining gold nanoclusters used in the system.

Another objective of this invention is to realize a system which is used to ensure that treatment can be started early by being used by the first responders and to inform the healthcare personnel about the cyanide content in the biological sample so that the first response can be performed properly.

Another objective of this invention is to realize a system which ensures that the cyanide determination can be easily performed in the biological samples by all healthcare professionals, except expert personnel, after a short training.

Another objective of the present invention is to realize a system that allows determination of the test results in a shorter time without having to wait for the results of the analysis, due to the fact that cyanide poisoning requires rapid intervention.

Another objective of this invention is to realize a determination system which is easy to use, portable, lightweight, quick and with low-cost instead of high-cost and heavy devices used for the cyanide determination in biological samples.

Another objective of this invention is to provide a system which enables to work with an amount of sample at a microliter level and ensures that the related sample can be directly used for determination without the need for pre-treatment such as acid treatment, storage and transportation before analysis.

Another objective of this invention is to provide a system which facilitates the decision on the type of treatment to be applied by making a distinction between the poisonings due to the similar symptoms that occur in cyanide and carbon monoxide poisonings, and enables the right treatment process to be started quickly.

Another objective of this invention is to provide a system and method which enables to obtain gold nanoclusters emitting fluorescent color and to reach the information about the cyanide concentration in the sample by examining the color fadings that occur in the fluorescent color on the basis of the cyanide concentration during their use in the system.

### DETAILED DESCRIPTION OF THE INVENTION

“A Cynadide Determination System and a Method for Obtaining Gold Nanoclusters Used in the System” realized to fulfil the objectives of the present invention is shown in the figures attached, in which:

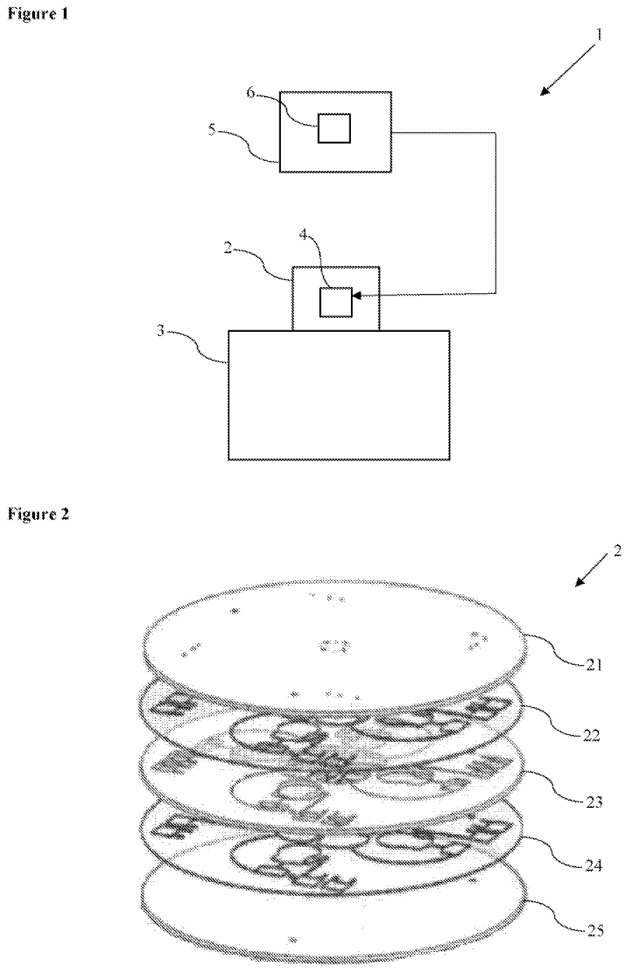

FIG. 1 is a schematic view of the inventive cyanide determination system.

FIG. 2 is a view illustrating the layers of the compact disc included in the inventive system.

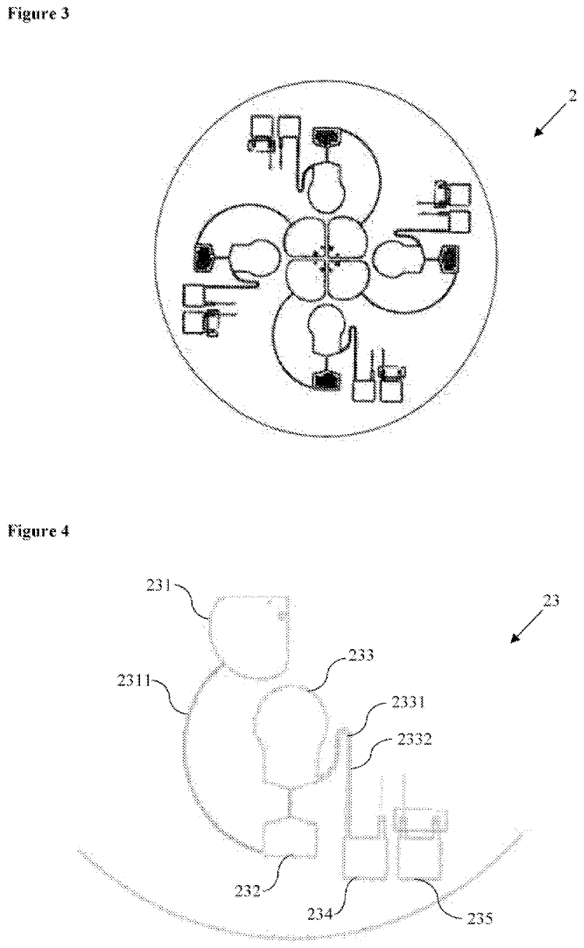

FIG. 3 is a top view of the compact disc included in the inventive system.

FIG. 4 is a view illustrating the areas on the compact disc included in the inventive system.

FIG. 5 is a graph related to the RPM and time intervals which are applied for the plasma separation process on the compact disc included in the inventive system.

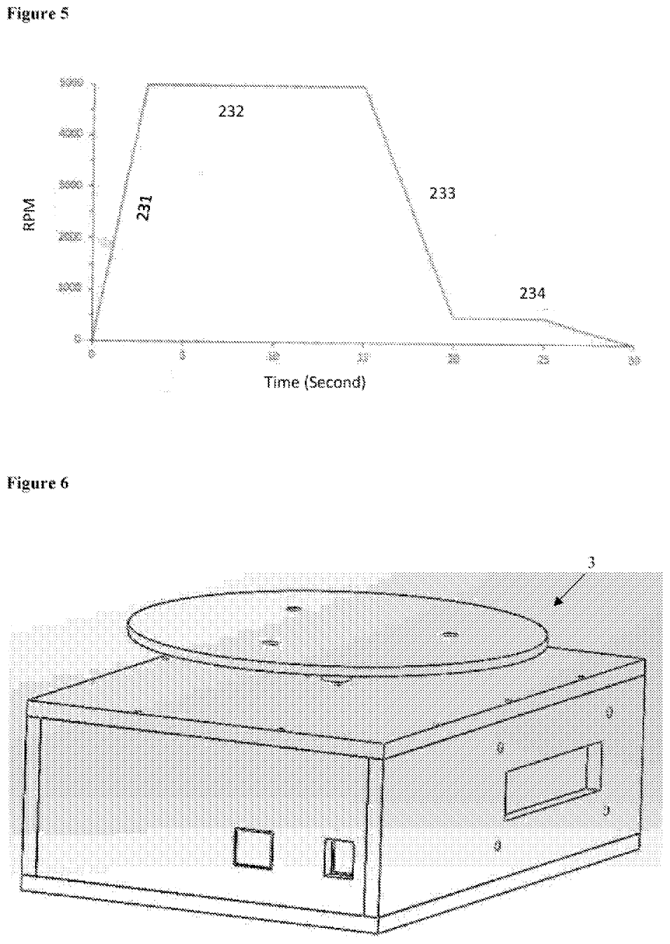

FIG. 6 is a view of the centrifuge device included in the inventive system.

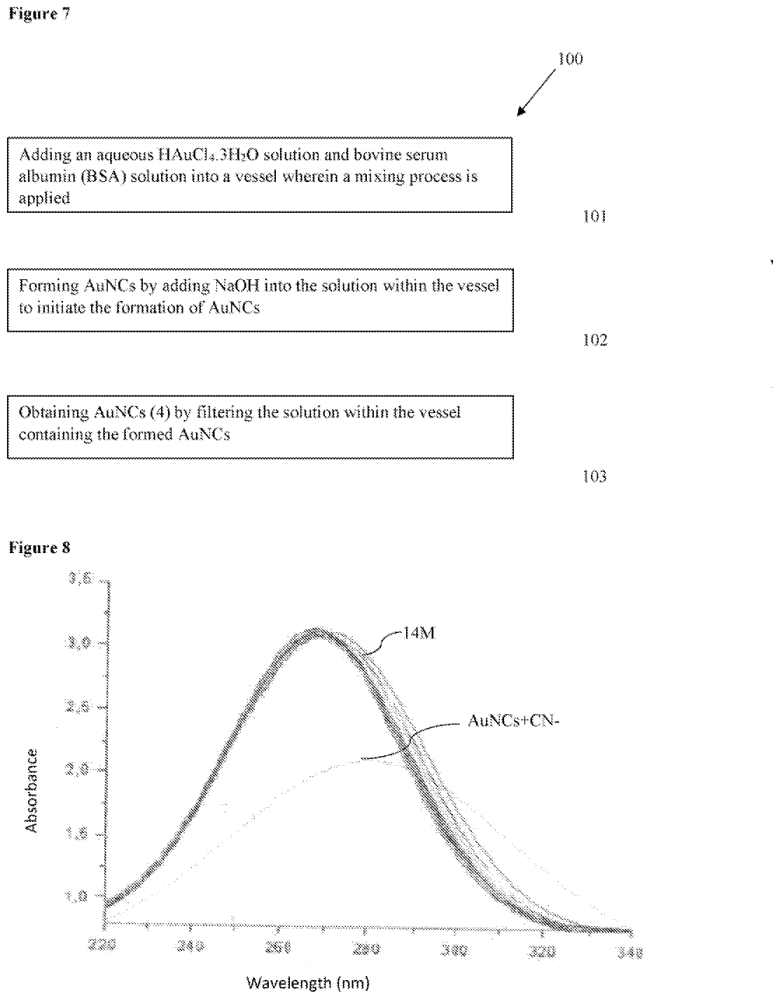

FIG. 7 is a flow chart of the inventive method for obtaining gold nanocluster.

FIG. 8 is a view of the UV absorbance spectrum results of the inventive gold nanoclusters alone and in the presence of anions.

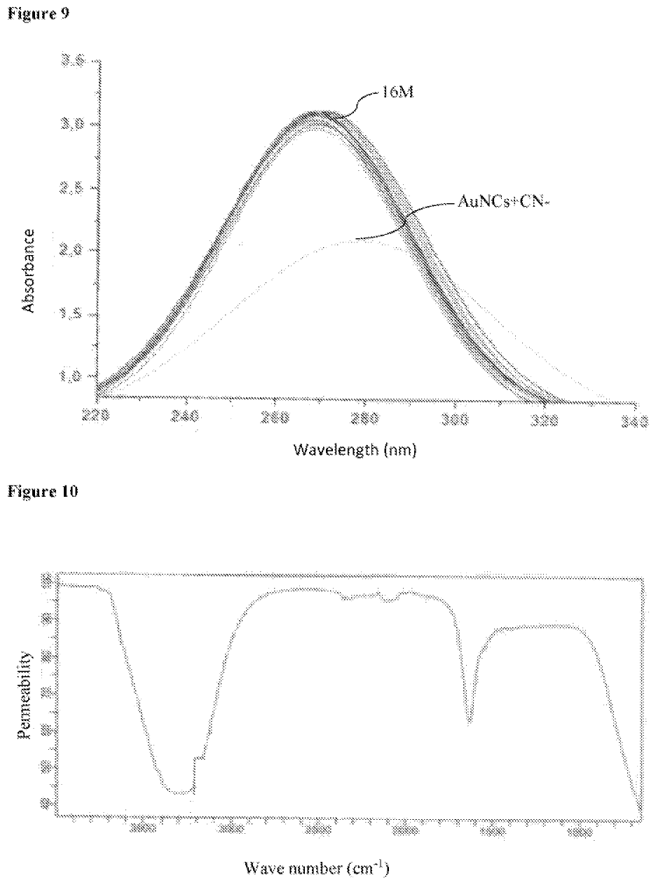

FIG. 9 is a view of the UV absorbance spectrum results of the inventive gold nanoclusters alone and in the presence of metal ions.

FIG. 10 is a view of the ATR-FTIR spectrum of the BSA stabilized AuNCs.

FIG. 11 is a view of the ATR-FTIR spectrum of the BSA solution.

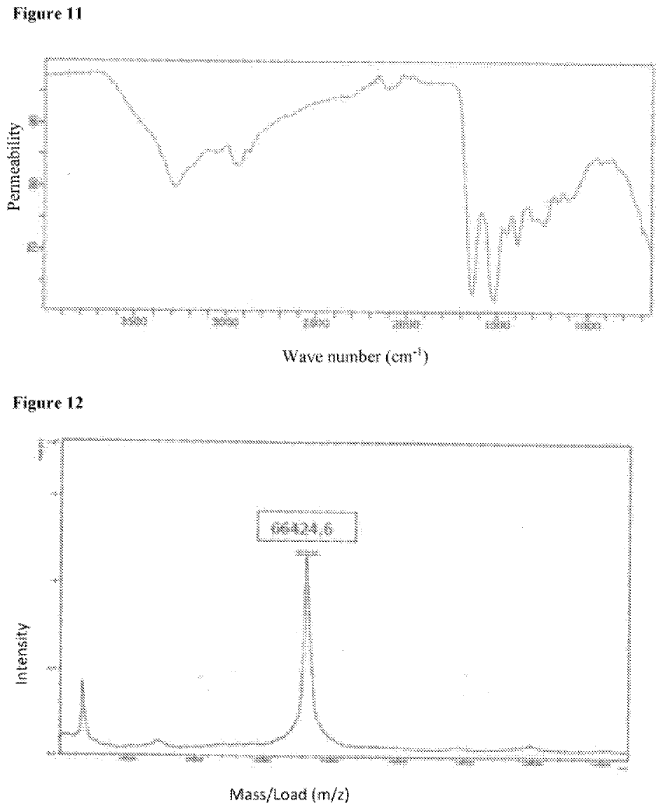

FIG. 12 is a view of the MALDI-TOF-MS spectrum of the BSA.

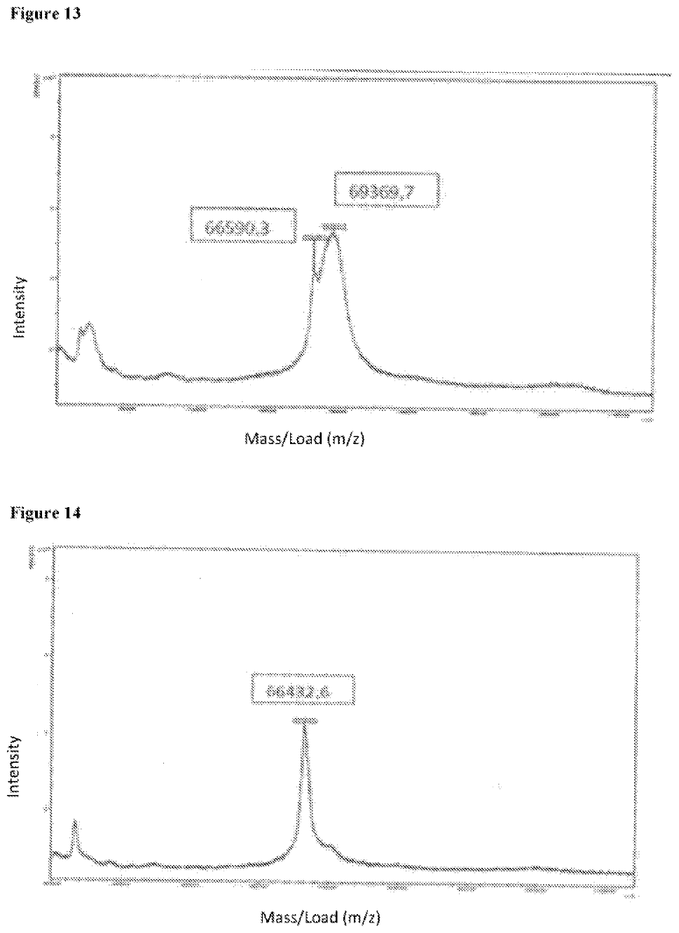

FIG. 13 is a view of the MALDI-TOF-MS spectrum of the BSA stabilized AuNCs.

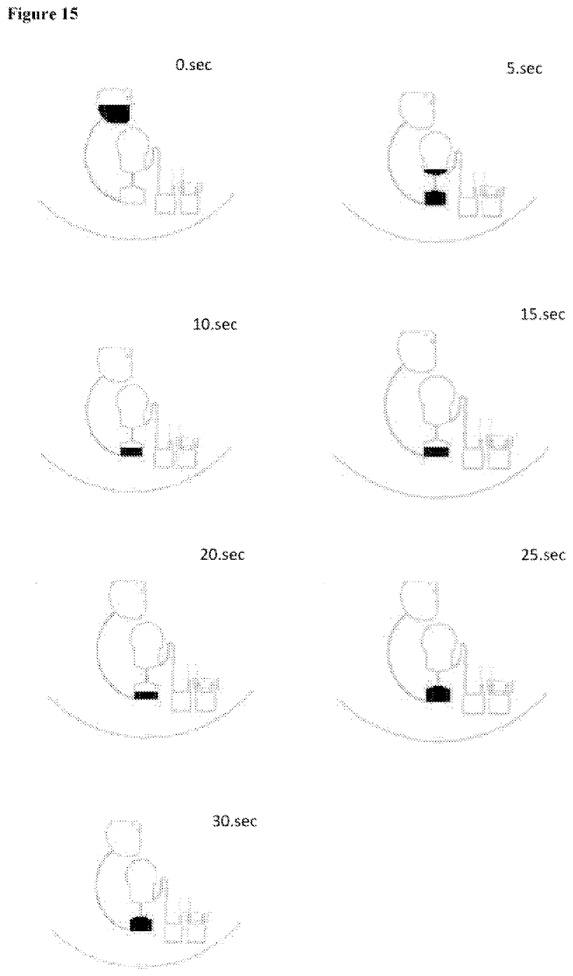

FIG. 14 is a view of the MALDI-TOF-MS spectrum of the cyanide-treated BSA-stabilized AuNCs within the dialysis membrane.

FIG. 15 is real-time images of the sample separation process which is carried out by using the inventive compact disc.

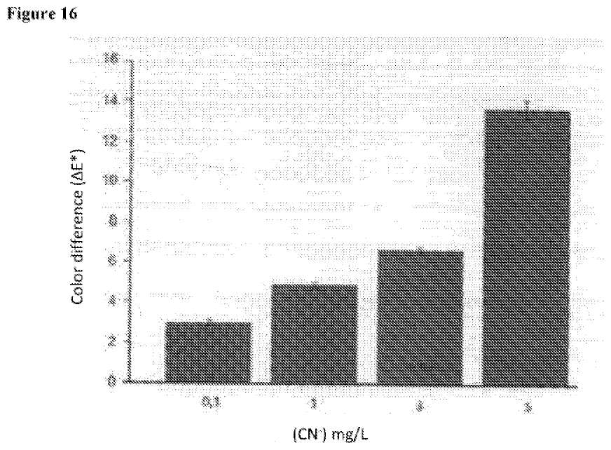

FIG. 16 is a graph which is created for the color difference values obtained on the AuNCs depending on the amount of CN⁻.

The components illustrated in the figures are individually numbered, where the numbers refer to the following:
1. System
2. Compact Disc (CD)
   21. Polymethylmethacrylate (PMMA) upper layer
   22. First adhesive layer
   23. Polymethylmethacrylate (PMMA) middle layer
      231. Filling chamber
         2311. First channel
      232. Separation chamber
      233. Pneumatic chamber
         2331. Second channel
         2332. Pneumatic siphon valve
      234. Plasma collection chamber
      235. Comparison chamber
   24. Second adhesive layer
   25. Polymethylmethacrylate (PMMA) lower layer
3. Centrifuge device
4. Gold nanocluster (AuNC)
5. Electronic device
6. Application

The inventive system (1) which enables to carry out cyanide determination in biological samples taken from the living beings who are exposed to cyanide gas and/or cyanide components released from the industry and/or house fires comprises:
at least one microfluidic compact disc (CD) (2) which consists of at least one layer having at least one channel and at least one chamber having an area wherein the plasma of a blood sample, which is a biological sample, can be filled to be separated;
at least one centrifuge device (3) whereon the compact disc (2) is placed and which enables the centrifugation process that is required to separate the blood sample in the compact disc (2) into its plasma;
a gold nanocluster (AuNC) (4) which is located in the compact disc (2), has a fluorescent color-emitting property, and the fluorescent color of which can fade at different rates according to the cyanide concentration by interacting with the cyanide in the plasma when it is combined with the plasma separated from the cyanide-containing blood sample;
at least one electronic device (5) which is configured to enable to capture image for the purpose of determination of fluorescent colors of the combination of gold nanoclusters (4) and the cyanide-containing plasma under UV light in order to determine the amount of cyanide in the blood plasma under examination; and
at least one application (6) which is executed on the electronic device (5) and configured to determine the amounts of cyanide corresponding to the colors in the images captured by the electronic device (5).

The compact disc (2) included in the inventive system (1) is configured to separate the plasma from the blood so as to detect the cyanide—which is a biological sample—in the blood. In a preferred embodiment of the invention, the compact disc (2) comprises three separate layers, and two layers with adhesive properties which are located between these layers and enable to join the layers with each other and to make them uniform. The compact disc (2) comprises three polymethylmethacrylate (PMMA) layers—preferably the top (21), the middle (23) and the bottom (25) one- and double-sided tape layers with adhesive properties—namely, the first adhesive (22) and the second adhesive (24)—to be placed between these layers (21, 23, 25). After superimposing the layers (21-22-23-24-25), a pressing process is applied to ensure that five layers can be joined with each other. In a preferred embodiment of the invention, this pressing process takes 36 hours. In a preferred embodiment of the invention, the upper, the middle and the lower PMMA layers (21, 23, 25) of the compact disc (2) have a thickness of 1.5 mm and a diameter of 120 mm. In a preferred embodiment of the invention, the first and the second adhesive layers (22, 24) of the compact disc (2) have a thickness of 130 m and a diameter of 120 mm.

The bottom PMMA layer (25) of the compact disc (2) comprises no functional channels or holes and serves as a supporting platform for the other layers.

The middle PMMA layer (23) of the compact disc (2) has five different chambers-namely, the filling chamber (231), the separation chamber (232), the pneumatic chamber (233), the plasma collection chamber (234) and the comparison chamber (235)—and a first channel (2311) that carries the whole blood from the filling chamber (231) to the separation chamber (232), a second channel (2331) that carries the plasma, which passes through the channel extending from the separation chamber (232) to the pneumatic chamber (233) during the centrifugation process for the separation process, from the pneumatic chamber (233) to the plasma collection chamber (234) and a pneumatic siphon valve (2332) that is located between the pneumatic chamber (233) and the plasma collection chamber (234). The filling chamber (231), which is located in the middle PMMA layer (23) of the compact disc (2), is configured to be the place wherein the sample desired to be analyzed is dripped in order to determine the cyanide content. The separation chamber (232), which is located in the middle PMMA layer (23) of the compact disk (2), is configured to be the place wherein the sample to be analyzed is sent before the centrifugation process. The pneumatic chamber (233), which is located in the middle PMMA layer (23) of the compact disc (2), is configured to be the place wherein the plasma separated from the blood, which is the biological sample, is collected during the centrifugation process of the sample to be analyzed. The plasma collection chamber (234), which is located in the middle PMMA layer (23) of the compact disc (2), is configured to be the place wherein the plasmas that are collected in the pneumatic chamber (233) by being separated from the blood by AuNCs are mixed with each other. The plasma collection chamber (234) contains the AuNC solution and an empty volume for the plasma leaving the blood to come in. In a preferred embodiment of the invention, the plasma collection chamber (234), which is located in the middle PMMA layer (23) of the compact disc (2), has a total volume of 100 μL and contains an AuNC solution of 50 μL. The comparison chamber (235), which is located in the middle PMMA layer (23) of the compact disc (2), is configured to contain a mixture of buffer solution and AuNC solution. In a preferred embodiment of the invention, the comparison chamber (235), which is located in the middle PMMA layer (23) of the compact disc (2), contains a total of 100 μL of 1:1 mixed phosphate buffered saline solution (PBS) and AuNC solution. The comparison chamber (235), which is located in the middle PMMA layer (23) of the compact disc (2), is configured to enable the composition of plasma and AuNC in the plasma collection chamber (234) to be compared with the fluorescent color emitted by AuNCs that do not comprise plasma. In a preferred embodiment of the invention, the first channel (2311), which is located in the middle PMMA layer (23) of the compact disc (2) and enables whole blood to be carried from the filling chamber (231) to the separation chamber (232), has a width of 200 μm. In a preferred embodiment of the invention, the second channel (2331), which is located in the middle PMMA layer (23) of the compact disc (2) and carries the plasma separated from the blood from the pneumatic chamber (233) to the plasma collection chamber (234), has a width of 500 μm. The pneumatic siphon valve (2332) is configured to prevent the blood from escaping from the pneumatic chamber (233) and passing into the plasma collecting chamber (234) through the siphon formed by the centrifugal force that is generated during the centrifugation process.

The bottom PMMA layer (25) of the compact disc (2) comprises holes that allow the blood sample to be filled for cyanide detection, and ventilation holes in order to adjust the required pressure and to prevent the formation of air bubbles during filling.

The centrifuge device (3) included in the inventive system (1) comprises a CD holder tray wherein the biological sample in the compact disc (2) is placed for centrifugation, and a structure preferably having a form of rectangular plasma under the tray (FIG. 6). The centrifuge device (3) has the properties of portability, lightness, and not spacious and it can be connected to a computer and controlled via a USB cable. The centrifuge device (3) can be adjusted and programmed to a rotation speed in the range of 500-6500 RPM by means of the control unit. The centrifuge device (3) is a device having an Ardino UNO circuit board and a power supply.

The electronic device (5) included in the inventive system (1) is device such as smart phone, tablet, desktop computer or portable computer whereon at least one application (6) can be executed, which has at least one camera that enables image capturing, and has an input unit in the form of a key or touch screen. The electronic device (5) enables the images of cyanide-containing samples, which are centrifuged in the compact disc (2) and emit different fading colors according to the cyanide concentration by combining with AuNCs, to be captured under UV light. The electronic device (5) enables the images taken from the samples to be transmitted to the application (6) in order to determine the cyanide concentrations.

The application (6) included in the inventive system (1) is executed on the electronic device (5) and determines the cyanide concentration in the sample from the color data in the image by ensuring that the images captured by the electronic device (5) are evaluated according to the colors stored in the database within the application (6) and the cyanide reference values corresponding to these colors. In an embodiment of the invention, the application (6) is the Color Grab application which presents values through the L*a*b* color system for color changes due to the fading mechanisms of AuNCs. L*a*b* color values have a coordinate system wherein each color is represented by a single point, just like the geographic coordinate system (latitude, longitude and altitude). Therefore, three components (color coordinate values) are required to define each color in the color space. Here, L* is the lightness coordinate (L*=0 denotes black and L*=100 denotes white), a* is the red/green coordinate (+a* denotes red, −a* denotes green), and b* is the yellow/blue coordinate (+b* denotes yellow, −b* denotes blue). For analyzing the color difference between two images or objects, the following equation is used:

$$Δ {E}_{a b}^{*} = √ {( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}$$

L*a*b color values are the most commonly used method in measurement and color communication. L*a*b* values are designed in analogous with human eye perception.

The said invention also comprises the method for obtaining gold nanoclusters (AuNC) (4).
100. Method

The inventive method (100) for obtaining gold nanocluster (AuNC) (4) which emits fluorescent color, wherein the fluorescent color fades in accordance with the cyanide concentration when interacting with cyanide, and which is used as the detection mechanism of the gold atoms of the cyanide, comprises the following steps:
adding an aqueous HAuCl₄.3H₂O solution and bovine serum albumin (BSA) solution into a vessel wherein a mixing process is applied (101);
forming AuNCs by adding NaOH into the solution within the vessel to initiate the formation of AuNCs (102); and
obtaining AuNCs (4) by filtering the solution within the vessel containing the formed AuNCs (103).

In the step of adding an aqueous HAuCl₄.3H₂O solution and bovine serum albumin (BSA) solution into a vessel wherein a mixing process is applied (101) of the inventive method (100); into a glass vessel, washed with aqua regia solution, rinsed with deionized water and dried; 5 ml of 50 mg/ml BSA solution used as stabilizing agent is added into 5 ml of 10 mM aqueous HAuCl₄.3H₂0 solution under vigorous stirring at 30-40° C. In another preferred embodiment of the invention, 5 ml of 50 mg/ml BSA solution used as stabilizing agent is added into 5 ml of 10 mM aqueous HAuCl₄.3H₂O solution under vigorous stirring at 50° C.

In the step of forming AuNCs by adding NaOH into the solution within the vessel to initiate the formation of AuNCs (102) of the inventive method (100); NaOH is added into the solution in the vessel to adjust the pH and initiate AuNC formation. In a preferred embodiment of the invention, NaOH is added into the vessel containing the solution after 2 minutes. In another preferred embodiment of the invention, NaOH is added after 30 minutes. In a preferred embodiment of the invention, 0.5 ml of 1 M NaOH is added. The reaction formed after adding NaOH is continued for 12 hours under vigorous stirring at a temperature of 30-40° C. In another preferred embodiment of the invention, the reaction formed after adding NaOH is continued for 3-4 hours at a temperature of 50° C. under vigorous stirring.

In another preferred embodiment of the steps 101 and 102 of the inventive method (100), 19.2 mg mL⁻¹ BSA solution is added into 5.8 mmol L⁻¹ HAuCl₄.3H₂O solution and the mixing is carried out under vigorous stirring at a temperature of 22.5-30° C. After 2 minutes, 38 mmol L⁻¹ NaOH is added to adjust the acidity of the solution (so as to be preferably pH 10). After 5 minutes, the solution is incubated at 100° C. for 1 hour.

NaOH, which is used to adjust the pH of the solution during the reaction, causes conformational changes in the secondary structure of BSA and thus enables the Au atoms to be clustered (packaged) in BSA and therefore realizes the formation of AuNC. AuNCs interact covalently with the thiol (—SH) groups of cysteine residues on the BSA surface. In addition, Au(III) ions are reduced to Au(I) ions at high alkaline pH formed by NaOH that is added into the solution. The step of reducing from Au(I) to Au(0) is provided by the tyrosine residues on the BSA. Non-functional disulfide bonds in the BSA secondary structure at low pHs become suitable for forming strong Au—S bonds at high alkaline pH and enables the BSA to form and hold AuNCs together by wrapping the gold atoms.

In the step of obtaining AuNCs (4) by filtering the solution within the vessel containing the formed AuNCs (103) of the inventive method (100); it is understood that the synthesis is completed when the color of the reaction solution changes from light yellow to brown and the solution is filtered through a filter with 0.2-0.5 μm pore size and stored at 4° C. or frozen to obtain pure BSA-AuNCs powder.

The shape and size analysis of the BSA-stabilized AuNCs prepared, changes of absorbance intensity after interaction with cyanide and different materials for selectivity studies, structural changes, the average atomic numbers per cluster and fading mechanisms in the presence of cyanide were analyzed by HR-TEM, UV-Vis spectroscopy, ATR-FTIR, and MALDI-TOF-MS spectra, respectively.

BSA-stabilized AuNCs were analyzed to be spherical with 3-5 nm size distribution as a result of HR-TEM analysis. In the HR-TEM image at 10 nm scale, clusters are mostly seen due to particle coalescence under electron beam, whereas the shape structure of AuNCs at 5 nm scale can be observed more clearly.

The data of 15 different anion substances (14M) in total, including AuNCs, (AuNCs, AuNCs+S²⁺, AuNCs+Ac⁻, AuNCs+EDTA²⁻, AuNCs+NO₂⁻, AuNCs+Sitrat, AuNCs+Cl⁻, AuNCs+NO²⁻, AuNCs+N₂⁺, AuNCs+PO₄³⁻, AuNCs+C₂O₄²⁻, AuNCs+F⁻, AuNCs+SO₄²⁻, AuNCs+CO₃²⁻) in the UV-Vis spectrum and of AuNCs+CN⁻ are given in the graph in the FIG. 8. As can be seen from the graph, cyanide (CN⁻) causes a decrease of approximately 1.1 units in the absorbance value of AuNCs. It is observed that 13 different anions other than cyanide do not cause a significant change on the absorbance intensity of AuNCs. Also, the data of 16 different metal ion substances (16M) in total, including AuNCs, (AuNCs, AuNCs+Ni²⁺, AuNCs+FE³⁺, AuNCs+Cd²⁺, AuNCs+Pb²⁺, AuNCs+K⁺, AuNCs+Li⁺, AuNCs+Mg²⁺, AuNCs+Ca²⁺, AuNCs+Ba²⁺, AuNCs+Al³⁺, AuNCs+Karişim_{[K+, Li+, Mg2+, Ca2+, Ba2+ve Al3+]}, AuNCs+An²⁺, AuNCs+Co²⁻, AuNCs+Cu²⁺, AuNCs+Hg²⁺) in the UV-Vis spectrum and of AuNCs+CN⁻ are given in the graph in the FIG. 9. As can be seen from the graph, cyanide causes a decrease of approximately 1.1 units in the absorbance intensity of AuNCs. It is observed that among the solution consisting of a mixture of 14 different metal ions and metal ions other than cyanide, only Hg2+, Cu2+, Co2+, and Zn2+ ions cause a 0.10-0.15 unit change in the absorbance intensity of AuNCs. In the experiment, 2,6-pyridinedicarboxylic acid (PDCA), BSA and Glutathione (GSH) are used for chelation.

ATR-FTIR spectra of BSA-stabilized fluorescent AuNCs and only BSA solution are shown in the FIG. 10 and the FIG. 11. Accordingly, the band seen as 3280 cm⁻¹ is the O—H/N—H stretching frequency of BSA. Vibration bands of BSA are still present in BSA-stabilized AuNCs, although a reduction occurs in its intensity. The peak at 1660 cm⁻¹ is the characteristic amide band mainly due to C═O stretching vibrations, as well as the out-of-phase C≡N stretching vibrations. The amide I band region between 1600-1700 cm⁻¹ gives more information about the secondary alpha-helix structure of the protein. The slight broadening of the amide I band represents the change in the secondary structure of the protein that occurs during the formation of BSA-stabilized AuNCs.

MALDI-TOF-MS analyzes were performed to determine the interactions of the prepared AuNCs with cyanide. For the measurements, in order to remove the salts in the prepared AuNCs solution, it was placed in the dialysis membrane with a molecular weight separation limit of 10 kDa and dialyzed in deionized water for 24 hours (the deionized water was changed every 6 hours). There is shown MALDI-TOF-MS spectrum of BSA in the FIG. 12, MALDI-TOF-MS spectrum of BSA-stabilized AuNCs in the FIG. 13 and MALDI-TOF-MS spectrum of cyanide-treated BSA-stabilized AuNCs in the dialysis membrane in the FIG. 14. When AuNCs, which are trapped in BSA and cannot pass through the dialysis membrane under normal conditions, are removed from the inside of the BSA by the effect of cyanide; they pass through the dialysis membrane due to their molecular weight and mix with the deionized water outside the membrane. In the Maldi-Tof-Ms spectrum, it is seen that the signal of BSA-stabilized AuNCs after cyanide interaction reverts to its initial state wherein there is almost no cyanide in the medium. In addition, the BSA signal with a molecular weight of 66.4 kDa reaches a molecular weight of 69.4 kDa after the formation of AuNCs and a 3 kDa shift occurs in the spectrum. When this shift value in the spectrum is proportional to the gold atomic mass value (0.1969 kDa), it is understood that each BSA stabilized AuNC consists of 15 gold atoms.

In order to perform a real-time plasma separation on the compact disc (2), 200 μL of CN-treated whole blood sample is filled into the filling chamber (231) (0 sec). Upon the CD (2) reaches 5000 RPM speed in approximately 3 seconds, the whole blood in the filling chamber (231) is pushed towards the separation chamber (232) by centrifugal force (5 sec). The pneumatic chamber (233) and the plasma collection chamber (234) are connected to each other by a channel of 200 μm width and by a centrifugal-pneumatic siphon valve (2332). Since CD accelerates to 5000 RPM in a very short time, the centrifugal thrust force prevents the blood from flowing through this valve (2332) and keeps the blood at a safe distance close to the elbow area. The blood filled into the separation chamber (232) begins to separate into its components and compresses the air in the pneumatic chamber (233) located above it (10 sec-15 sec). The centrifugal force on the CD rotating at 5000 RPM until the 15^{th} second, is greater than the force that it exerts on the blood separated into its components under the compressed air in the pneumatic chamber (233). After 15 seconds, the CD rotation speed is reduced to 500 RPM. Thus, the air compressed in the pneumatic chamber (233) begins to expand and pushes the blood under it towards the siphon (20 sec). It is waited for 10 seconds for 50 μL of the blood separated to its components to completely fill into the plasma collection chamber (234) and for the siphoning process to end (25 sec-30 sec) (FIG. 15). The color differences (ΔE*) of AuNCs treated with cyanide at different concentrations, which were separated on the microfluidic CD, are extracted by the Color Grab application (6). The most accurate results are obtained by extracting the base color difference value (1.58) obtained by interacting the cyanide-free plasma liquid with AuNCs. The graph of the color difference values obtained depending on the amount of CN— is given in the FIG. 16.

According to the information in the literature, 0.5-1 mg/L cyanide in the blood leads to some minor symptoms in the body. While 2-3 mg/L of cyanide in the blood causes severe symptoms in the body, amounts greater than 3 mg/L usually result in death. As a result of the obtained results, in plasma samples separated on CD in comparison to cyanide-containing plasma samples separated by a standard centrifuge device (3), it is observed that a more precise measurement range can be determined according to the cyanide concentration differences and high accuracy measurements can be made between 0.5 mg/L and 5 mg/L cyanide concentrations, which are harmless to human health/cause death. Thus, a system that allows the cyanide determination 5 times below the 0.5 mg/L cyanide concentration, which is mentioned in the literature and causes some minor symptoms in the body, has been developed. Due to the fact that the system enabling blood separation on microfluidic CD separates the plasma with higher purity compared to manual and pipette separation; it can be said that cyanide interaction with AuNCs is carried out more efficiently. It is also known that the UV permeability of PMMA used in the compact disc (2) is much higher compared to polypropylene (PP) that is a material from which Eppendorf tubes used for blood separation are produced. As a result, it has been proven that the use of PMMA for similar microfluidic CD systems to be used for the determination system under UV light source gives more efficient results. In accordance with this information, it is proven that the cyanide determination process from whole blood plasma can be performed with fluorescent AuNCs on a PMMA layered microfluidic CD by using a smartphone and the Color Grab application (6).

The inventive system (1) can be used to determine cyanide in biological samples such as blood, as well as to perform a fast and reliable determination in exposures originating from military or chemical weapons and to determine cyanide in drinking water or water used for irrigation of crops, especially in the agricultural sector, and in gold mines during gold digging processes with cyanide.

The inventive system (1) provides an easy-to-use and low-cost cyanide determination system that can be studied with a sample amount at the microliter level and does not require additional processes such as acid treatment, sample storage and transportation as often seen in traditional determination methods. Further, this system (1)—which is extremely fast and cost-effective in terms of analysis time that plays the biggest role in the treatment of cyanide poisoning-enables to minimize deaths due to cyanide poisoning. While cyanide can be determined from whole blood plasma within 25 minutes with the system (1), this period is approximately one and a half hours with the GC-MS device which is frequently used among traditional laboratory methods for the cyanide determination from whole blood (for example, with acid treatment, centrifugation, heating and cooling). Also, this period may increase depending on the occupation condition of the GC-MS device and the health personnel authorized to use the device. In addition, the GC-MS device is not available in every hospital located in smaller residential areas. Therefore, for example, it should be sent to more extensive health institutions and analyzed. And this causes the cyanide determination period to be prolonged. The said system (1) can centrifuge blood with high purity to be able to determine cyanide from blood plasma, and its total weight is approximately 0.5 kg. It also has a lower cost compared to devices used for traditional blood separation methods. The system can be easily used with a short training provided to the health personnel for the cyanide determination process to be carried out with the system (1). Also, the system (1) costs tens of times less than the traditional laboratory equipment used. The system (1) can be used for diagnostic purposes at the bedside and informs the health personnel so that the treatment can be started early and the first response can be done properly.

Within these basic concepts; it is possible to develop various embodiments of the inventive “A Cynadide Determination System and a Method for Obtaining Gold Nanoclusters Used in the System; the invention cannot be limited to examples disclosed herein and it is essentially according to claims.

1. A system (1) which enables to carry out cyanide determination in biological samples taken from the living beings who are exposed to cyanide gas and/or cyanide components released from the industry and/or house fires; characterized in that it comprises:
at least one microfluidic compact disc (CD) (2) which consists of at least one layer having at least one channel and at least one chamber having an area wherein the plasma of a blood sample, which is a biological sample, can be filled to be separated;
at least one centrifuge device (3) whereon the compact disc (2) is placed and which enables the centrifugation process that is required to separate the blood sample in the compact disc (2) into its plasma;
a gold nanocluster (AuNC) (4) which is located in the compact disc (2), has a fluorescent color emitting property, and the fluorescent color of which can fade at different rates according to the cyanide concentration by interacting with the cyanide in the plasma when it is combined with the plasma separated from the cyanide-containing blood sample;
at least one electronic device (5) which is configured to enable to capture image for the purpose of determination of fluorescent colors of the combination of gold nanoclusters (4) and the cyanide-containing plasma under UV light in order to determine the amount of cyanide in the blood plasma under examination; and
at least one application (6) which is executed on the electronic device (5) and configured to determine the amounts of cyanide corresponding to the colors in the images captured by the electronic device (5).

2. (canceled)

3. (canceled)

4. A system (1) according to claim 1; characterized by the compact disc (2) which comprises three polymethylmethacrylate (PMMA) layers—namely the top (21), the middle (23) and the bottom (25) one- and double-sided tape layers with adhesive properties—namely, the first adhesive (22) and the second adhesive (24)—to be placed between these layers (21, 23, 25).

5. (canceled)

6. (canceled)

7. (canceled)

8. A system (1) according to claim 4; characterized by the compact disc (2) which comprises the middle PMMA layer (23) with five different chambers-namely, the filling (231), the separation (232), the pneumatic (233), the plasma collection (234) and the comparison chamber (235).

9. (canceled)

10. A system (1) according to claim 8; characterized by the compact disc (2) which comprises the middle PMMA layer (23) with a second channel (2331) that carries the plasma-passing through the channel from the separation chamber (232) to the pneumatic chamber (233) during the centrifugation process being carried out for the separation process—from the pneumatic chamber (233) to the plasma collection chamber (234).

11. (canceled)

12. A system (1) according to claim 8; characterized by the compact disc (2) which comprises the middle PMMA layer (23) with the filling chamber (231) configured to be the place wherein the sample desired to be analyzed is dripped in order to determine the cyanide content.

13. A system (1) according to claim 8; characterized by the compact disc (2) which comprises the middle PMMA layer (23) with the middle PMMA layer (23) configured to be the place wherein the sample to be analyzed is sent before the centrifugation process.

14. A system (1) according to claim 8; characterized by the compact disc (2) which comprises the middle PMMA layer (23) with pneumatic chamber (233) configured to be the place wherein the plasma separated from the blood, that is the biological sample, is collected during the centrifugation process of the sample to be analyzed.

15. A system (1) according to claim 8; characterized by the compact disc (2) which comprises the middle PMMA layer (23) with the plasma collection chamber (234) configured to be the place wherein the plasmas that are collected in the pneumatic chamber (233) by being separated from the blood by AuNCs are mixed with each other.

16. A system (1) according to claim 8; characterized by the compact disc (2) which comprises the middle PMMA layer (23) with the plasma collection chamber (234) containing the AuNC solution and an empty volume for the plasma leaving the blood to come in.

17. (canceled)

18. A system (1) according to claim 8; characterized by the compact disc (2) which comprises the middle PMMA layer (23) with the comparison chamber (235) configured to contain a mixture of buffer solution and AuNC solution.

19. (canceled)

20. A system (1) according to claim 8; characterized by the compact disc (2) which comprises the middle PMMA layer (23) with the comparison chamber (235) configured to enable the composition of plasma and AuNC in the plasma collection chamber (234) to be compared with the fluorescent color emitted by AuNCs that do not comprise plasma.

21. (canceled)

22. (canceled)

23. A system (1) according to claim 8; characterized by the compact disc (2) which has the bottom PMMA layer (25) comprising holes that allow the blood sample to be filled for cyanide detection, and ventilation holes in order to adjust the required pressure and to prevent the formation of air bubbles during filling.

24. (canceled)

25. (canceled)

26. (canceled)

27. (canceled)

28. (canceled)

29. A system (1) according to claim 1; characterized by the electronic device (5) which enables the images of cyanide-containing samples, that are centrifuged in the compact disc (2) and emit different fading colors according to the cyanide concentration by combining with AuNCs, to be captured under UV light.

30. (canceled)

31. A system (1) according to claim 1; characterized by the application (6) which is executed on the electronic device (5) and determines the cyanide concentration in the sample from the color data in the image by ensuring that the images captured by the electronic device (5) are evaluated according to the colors stored in the database within the application (6) and the cyanide reference values corresponding to these colors.

32. A system (1) according to claim 31; characterized by the application (6) which is the Color Grab application which presents values through the L*a*b* color system for color changes due to the fading mechanisms of AuNCs.

33. A method (100) for obtaining gold nanocluster (AuNC) (4) which emits fluorescent color, wherein the fluorescent color fades in accordance with the cyanide concentration when interacting with cyanide, and which is used as the detection mechanism of the gold atoms of the cyanide; characterized in that it comprises the steps of:
adding an aqueous HAuCl4.3H2O solution and bovine serum albumin (BSA) solution into a vessel wherein a mixing process is applied (101);
forming AuNCs by adding NaOH into the solution within the vessel to initiate the formation of AuNCs (102); and
obtaining AuNCs (4) by filtering the solution within the vessel containing the formed AuNCs (103).

34. A method (100) according to claim 33; characterized in that in the step of adding an aqueous HAuCl4.3H2O solution and bovine serum albumin (BSA) solution into a vessel wherein a mixing process is applied (101) of the inventive method (100); into a glass vessel, washed with aqua regia solution, rinsed with deionized water and dried; 5 ml of 50 mg/ml BSA solution used as stabilizing agent is added into 5 ml of 10 mM aqueous HAuCl4.3H2O solution under vigorous stirring at 30-40° C.

35-43. (canceled)

44. A method (100) according to claim 33; characterized in that the NaOH, which is used to adjust the pH of the solution during the reaction, causes conformational changes in the secondary structure of BSA and thus enables the Au atoms to be clustered (packaged) in BSA and therefore realizes the formation of AuNC.

45-48. (canceled)

49. A method (100) according to claim 33; characterized in that in the step of obtaining AuNCs (4) by filtering the solution within the vessel containing the formed AuNCs (103); it is understood that the synthesis is completed when the color of the reaction solution changes from light yellow to brown and the solution is filtered through a filter with 0.2-0.5 m pore size and stored at 4° C. or frozen to obtain pure BSA-AuNCs powder.

50. A system (1) wherein the AuNCs obtained by following the step of the method (100), which is disclosed in claim 33, is used.
